(19) United States
(12) Patent Application Publication
(10) Pub. No.: US 2014/0257107 A1
(43) Pub. Date: Sep. 11, 2014

(54) Transducer Assembly for an Imaging Device

(71) Applicant: Volcano Corporation, San Diego, CA (US)

(72) Inventor: Cheryl Rice, San Diego, CA (US)

(73) Assignee: Volcano Corporation, San Diego, CA (US)

(21) Appl. No.: 14/103,330

(22) Filed: Dec. 11, 2013

Related U.S. Application Data
(60) Provisional application No. 61/747,153, filed on Dec. 28, 2012.

(51) Int. Cl. A61B 8/00

(52) U.S. Cl. CPC A61B 8/4494
USPC 600/459

(57) ABSTRACT

The present disclosure provides a transducer assembly. The transducer assembly includes a flex circuit. The transducer assembly also includes a first substrate that includes a piezoelectric micro-machined ultrasonic transducer (PMUT). The transducer assembly further includes a second substrate that includes an Integrated Circuit (IC) device. At least one of the first substrate and the second substrate is bonded to the flex circuit through wire bonding or through flip-chip.

### CROSS-REFERENCE TO RELATED APPLICATIONS

This application claims priority to Provisional Patent Application No. 61/747,153, filed Dec. 28, 2012, and entitled “Transducer Assembly for an Imaging Device,” the disclosure of which is hereby incorporated by reference in its entirety.

### TECHNICAL FIELD

The present disclosure relates generally to ultrasound imaging, and in particular, to a piezoelectric micromachined ultrasound transducer (PMUT) assembly.

### BACKGROUND

Intravascular ultrasound (IVUS) imaging is widely used in interventional cardiology as a diagnostic tool for assessing a vessel, such as an artery, within the human body to determine the need for treatment, to guide intervention, and/or to assess its effectiveness. An IVUS imaging system uses ultrasound echoes to form a cross-sectional image of the vessel of interest. Typically, IVUS imaging uses a transducer on an IVUS catheter that both emits ultrasound signals (waves) and receives the reflected ultrasound signals. The emitted ultrasound signals (often referred to as ultrasound pulses) pass easily through most tissues and blood, but they are partially reflected as the result of impedance variation arising from tissue structures (such as the various layers of the vessel wall), red blood cells, and other features of interest. The IVUS imaging system, which is connected to the IVUS catheter by way of a patient interface module, processes the received ultrasound signals (often referred to as ultrasound echoes) to produce a cross-sectional image of the vessel where the IVUS catheter is located.

IVUS catheters typically employ one or more transducers to transmit ultrasound signals and receive reflected ultrasound signals. However, conventional methods and apparatuses for providing transducer assemblies may be limited and may lack flexibility. Therefore, while conventional methods and apparatuses for providing transducer assemblies are generally adequate for their intended purposes, they have not been entirely satisfactory in every aspect.

### SUMMARY

Ultrasounds transducers are used in Intravascular ultrasound (IVUS) imaging to help assess medical conditions inside a human body. The ultrasound transducer is implemented as a part of transducer assembly, which may also include an Integrated Circuit (IC) device. The present disclosure is directed to various types of transducer assemblies that offer improved flexibility and versatility that conventional transducer assemblies often lack. In various examples, the ultrasound transducer the IC device of the transducer assembly of the present disclosure are implemented on separate substrates and are electrically coupled together through a flex circuit, wire bonds, flip chip bonding, or soldering or welding.

One aspect of the present disclosure involves a transducer assembly. The transducer assembly includes: a flex circuit; a first substrate that includes a piezoelectric micro-machined ultrasonic transducer (PMUT); and a second substrate that includes an Integrated Circuit (IC) device; wherein at least one of the first substrate and the second substrate is bonded to the flex circuit through wire bonding.

Another aspect of the present disclosure involves a transducer assembly. The transducer assembly includes: a flex circuit; a first substrate that includes a piezoelectric micro-machined ultrasonic transducer (PMUT); and a second substrate that includes an Integrated Circuit (IC) device; wherein at least one of the first substrate and the second substrate is bonded to the flex circuit through flip-chip.

Yet another aspect of the present disclosure involves a transducer assembly. The transducer assembly includes: a support substrate; a first substrate that includes a piezoelectric micro-machined ultrasonic transducer (PMUT); and a second substrate that includes an Integrated Circuit (IC) device; wherein the first substrate and the second substrate are each bonded to the support substrate, and wherein the first substrate and the second substrate are electrically coupled together through wire bonding

Both the foregoing general description and the following detailed description are exemplary and explanatory in nature and are intended to provide an understanding of the present disclosure without limiting the scope of the present disclosure. In that regard, additional aspects, features, and advantages of the present disclosure will become apparent to one skilled in the art from the following detailed description.

### BRIEF DESCRIPTIONS OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a schematic illustration of an intravascular ultrasound (IVUS) imaging system according to various aspects of the present disclosure.

FIGS. 2-9 are various diagrammatic top and cross-sectional views of different embodiments of the transducer assembly according to various aspects of the present disclosure.

FIG. 2 is a simplified diagrammatic top view of a transducer assembly according to an embodiment of the present disclosure.

FIG. 3A is a simplified diagrammatic top view of a transducer assembly according to another embodiment of the present disclosure.

FIG. 3B is a simplified diagrammatic cross-sectional view of the transducer assembly of FIG. 3A.

FIG. 4A is a top view of a transducer assembly according to an embodiment of the present disclosure.

FIG. 4B is a bottom view of the transducer assembly of FIG. 4A.

FIG. 5 is a simplified diagrammatic cross-sectional view of a transducer assembly according to another embodiment of the present disclosure.

FIG. 6 is a simplified diagrammatic cross-sectional view of a transducer assembly according to another embodiment of the present disclosure.

FIG. 7 is a simplified diagrammatic cross-sectional view of a transducer assembly according to another embodiment of the present disclosure.

FIG. 8A is a simplified diagrammatic cross-sectional view of a transducer assembly according to another embodiment of the present disclosure.

FIG. 8B is a simplified diagrammatic cross-sectional view of a transducer assembly according to another embodiment of the present disclosure.

FIG. 8C is a simplified diagrammatic cross-sectional view of a transducer assembly according to another embodiment of the present disclosure.

FIG. 8D is a simplified diagrammatic cross-sectional view of a transducer assembly according to another embodiment of the present disclosure.

FIG. 9A is a simplified diagrammatic cross-sectional view of a transducer assembly according to another embodiment of the present disclosure.

FIG. 9B is a simplified diagrammatic cross-sectional view of a transducer assembly according to another embodiment of the present disclosure.

FIG. 9C is a simplified diagrammatic cross-sectional view of a transducer assembly according to another embodiment of the present disclosure.

FIG. 9D is a simplified diagrammatic cross-sectional view of a transducer assembly according to another embodiment of the present disclosure.

FIGS. 10A-10C illustrate diagrammatic perspective views of an embodiment of a transducer assembly according to various aspects of the disclosure.

FIG. 10A is a diagrammatic perspective view of a transducer assembly according to another embodiment of the present disclosure.

FIG. 10B is a diagrammatic perspective, cross-sectional view of the transducer assembly of FIG. 10A.

FIG. 10C is a diagrammatic perspective view of the transducer assembly of FIG. 10A from a different perspective.

FIG. 11 illustrates a diagrammatic cross-sectional view of an embodiment of a further embodiment of a transducer assembly.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, the present disclosure provides an ultrasound imaging system described in terms of cardiovascular imaging, however, it is understood that such description is not intended to be limited to this application, and that such imaging system can be utilized for imaging throughout the body. In some embodiments, the illustrated ultrasound imaging system is a side looking intravascular imaging system, although transducers formed according to the present disclosure can be mounted in other orientations including forward looking. The imaging system is equally well suited to any application requiring imaging within a small cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

There are primarily two types of catheters in common use today: solid-state and rotational. An exemplary solid-state catheter uses an array of transducers (typically 64) distributed around a circumference of the catheter and connected to an electronic multiplexer circuit. The multiplexer circuit selects transducers from the array for transmitting ultrasound signals and receiving reflected ultrasound signals. By stepping through a sequence of transmit-receive transducer pairs, the solid-state catheter can synthesize the effect of a mechanically scanned transducer element, but without moving parts. Since there is no rotating mechanical element, the transducer array can be placed in direct contact with blood and vessel tissue with minimal risk of vessel trauma, and the solid-state scanner can be wired directly to the imaging system with a simple electrical cable and a standard detachable electrical connector.

An exemplary rotational catheter includes a single transducer located at a tip of a flexible driveshaft that spins inside a sheath inserted into the vessel of interest. The transducer is typically oriented such that the ultrasound signals propagate generally perpendicular to an axis of the catheter. In the typical rotational catheter, a fluid-filled (e.g., saline-filled) sheath protects the vessel tissue from the spinning transducer and driveshaft while permitting ultrasound signals to freely propagate from the transducer into the tissue and back. As the driveshaft rotates (for example, at 30 revolutions per second), the transducer is periodically excited with a high voltage pulse to emit a short burst of ultrasound. The ultrasound signals are emitted from the transducer, through the fluid-filled sheath and sheath wall, in a direction generally perpendicular to an axis of rotation of the driveshaft. The same transducer then listens for returning ultrasound signals reflected from various tissue structures, and the imaging system assembles a two dimensional image of the vessel cross-section from a sequence of several hundred of these ultrasound pulse/echo acquisition sequences occurring during a single revolution of the transducer.

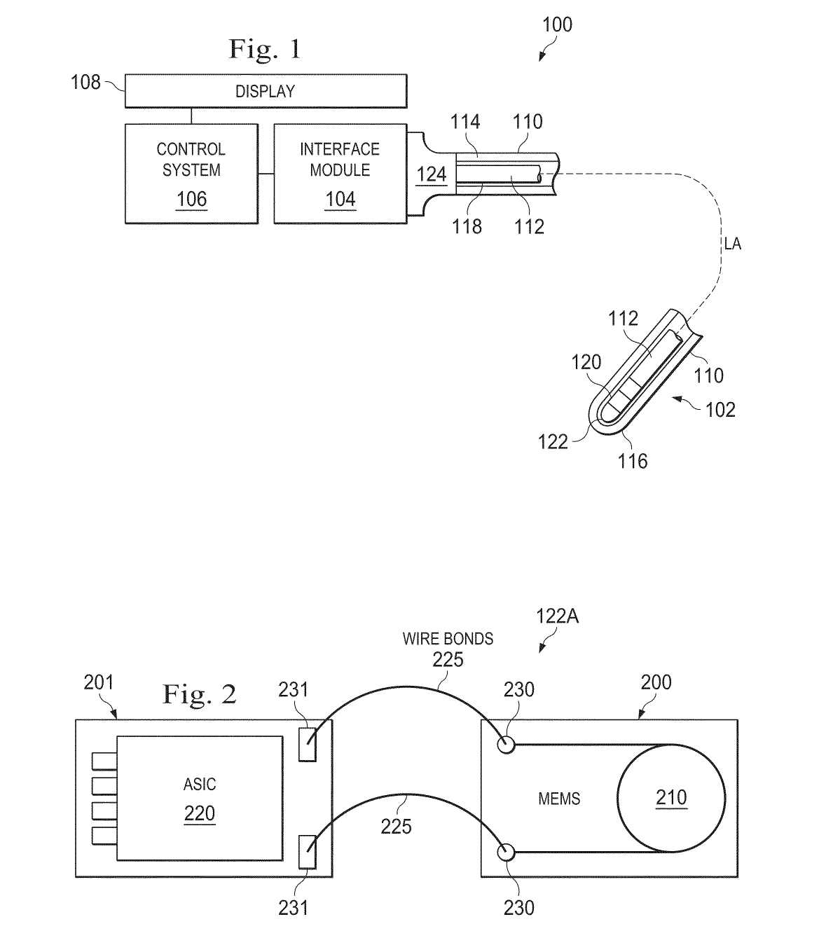

FIG. 1 is a schematic illustration of an ultrasound imaging system 100 according to various aspects of the present disclosure. In some embodiments, the ultrasound imaging system 100 includes an intravascular ultrasound imaging system (IVUS). The IVUS imaging system 100 includes an IVUS catheter 102 coupled by a patient interface module (PIM) 104 to an IVUS control system 106. The control system 106 is coupled to a monitor 108 that displays an IVUS image (such as an image generated by the IVUS system 100).

In some embodiments, the IVUS catheter 102 is a rotational IVUS catheter, which may be similar to a Revolution® Rotational IVUS Imaging Catheter available from Volcano Corporation and/or rotational IVUS catheters disclosed in U.S. Pat. No. 5,243,988 and U.S. Pat. No. 5,546,948, both of which are incorporated herein by reference in their entirety. The catheter 102 includes an elongated, flexible catheter sheath 110 (having a proximal end portion 114 and a distal end portion 116) shaped and configured for insertion into a lumen of a blood vessel (not shown). A longitudinal axis LA of the catheter 102 extends between the proximal end portion 114 and the distal end portion 116. The catheter 102 is flexible such that it can adapt to the curvature of the blood vessel during use. In that regard, the curved configuration illustrated in FIG. 1 is for exemplary purposes and in no way limits the manner in which the catheter 102 may curve in other embodiments. Generally, the catheter 102 may be configured to take on any desired straight or arcuate profile when in use.

A rotating imaging core 112 extends within the sheath 110. The imaging core 112 has a proximal end portion 118 disposed within the proximal end portion 114 of the sheath 110 and a distal end portion 120 disposed within the distal end portion 116 of the sheath 110. The distal end portion 116 of the sheath 110 and the distal end portion 120 of the imaging core 112 are inserted into the vessel of interest during operation of the IVUS imaging system 100. The usable length of the catheter 102 (for example, the portion that can be inserted into a patient, specifically the vessel of interest) can be any suitable length and can be varied depending upon the application. The proximal end portion 114 of the sheath 110 and the proximal end portion 118 of the imaging core 112 are connected to the interface module 104. The proximal end portions 114, 118 are fitted with a catheter hub 124 that is removably connected to the interface module 104. The catheter hub 124 facilitates and supports a rotational interface that provides electrical and mechanical coupling between the catheter 102 and the interface module 104.

The distal end portion 120 of the imaging core 112 includes a transducer assembly 122. The transducer assembly 122 is configured to be rotated (either by use of a motor or other rotary device) to obtain images of the vessel. The transducer assembly 122 can be of any suitable type for visualizing a vessel and, in particular, a stenosis in a vessel. In the depicted embodiment, the transducer assembly 122 includes a piezoelectric micromachined ultrasonic transducer (“PMUT”) transducer and associated circuitry, such as an application-specific integrated circuit (ASIC). An exemplary PMUT used in IVUS catheters may include a polymer piezoelectric membrane, such as that disclosed in U.S. Pat. No. 6,641,540, hereby incorporated by reference in its entirety. The PMUT transducer can provide greater than 100% bandwidth for optimum resolution in a radial direction, and a spherically-focused aperture for optimum azimuthal and elevation resolution.

The transducer assembly 122 may also include a housing having the PMUT transducer and associated circuitry disposed therein, where the housing has an opening that ultrasound signals generated by the PMUT transducer travel through. In yet another alternative embodiment, the transducer assembly 122 includes an ultrasound transducer array (for example, arrays having 16, 32, 64, or 128 elements are utilized in some embodiments).

The rotation of the imaging core 112 within the sheath 110 is controlled by the interface module 104, which provides user interface controls that can be manipulated by a user. The interface module 104 can receive, analyze, and/or display information received through the imaging core 112. It will be appreciated that any suitable functionality, controls, information processing and analysis, and display can be incorporated into the interface module 104. In an example, the interface module 104 receives data corresponding to ultrasound signals (echoes) detected by the imaging core 112 and forwards the received echo data to the control system 106. In an example, the interface module 104 performs preliminary processing of the echo data prior to transmitting the echo data to the control system 106. The interface module 104 may perform amplification, filtering, and/or aggregating of the echo data. The interface module 104 can also supply high- and low-voltage DC power to support operation of the catheter 102 including the circuitry within the transducer assembly 122.

In some embodiments, wires associated with the IVUS imaging system 100 extend from the control system 106 to the interface module 104 such that signals from the control system 106 can be communicated to the interface module 104 and/or visa versa. In some embodiments, the control system 106 communicates wirelessly with the interface module 104. Similarly, it is understood that, in some embodiments, wires associated with the IVUS imaging system 100 extend from the control system 106 to the monitor 108 such that signals from the control system 106 can be communicated to the monitor 108 and/or vice versa. In some embodiments, the control system 106 communicates wirelessly with the monitor 108.

As discussed above, the transducer assembly 122 includes a miniature ultrasound transducer and associated electronic circuitry. The transducer and the circuitry may be formed separately and later electrically interconnected together as a part of the transducer assembly 122. According to the various aspects of the present disclosure, several different embodiments of the transducer assembly 122 will now be discussed in more detail below.

FIG. 2 is a simplified diagrammatic top view of one embodiment of the transducer assembly 122A of the present disclosure. The transducer assembly 122A includes a micro-component 200 and a micro-component 201. In the illustrated embodiment, the micro-components 200-201 include micro-substrates and may thereafter be referred to as such. These micro-substrates have miniature dimensions, for example they may have a thickness ranging from about 75 microns (um) to about 600 um. In other embodiments, the micro-components 200-201 may include dies or other miniature devices suitable for the growth or placement of microelectronic devices.

An ultrasonic transducer 210 is formed on the micro-substrate 200. The ultrasonic transducer 210 has a small size and achieves a high resolution, so that it is well suited for intravascular imaging. In some embodiments, the ultrasonic transducer 210 has a size on the order of tens or hundreds of microns, can operate in a frequency range between about 1 mega-Hertz (MHz) to about 135 MHz, and can provide sub 50 micron resolution while providing depth penetration of up to 10 millimeters (mm). Furthermore, the ultrasonic transducer 210 is also shaped in a manner to allow a developer to define a target focus area based on a deflection depth of a transducer aperture, thereby generating an image that is useful for defining vessel morphology, beyond the surface characteristics. In the depicted embodiment, the ultrasound transducer 210 is a piezoelectric micromachined ultrasound transducer (PMUT). In other embodiments, the transducer 200 may include an alternative type of transducer. Additional details of the ultrasonic transducer 210 are described in Provisional U.S. Patent Application 61/745,091 to Dylan Van Hoven, filed on December 21, entitled “Preparation and Application of a Piezoelectric Film for an Ultrasound Transducer”, and attorney docket 44755.1060, and Provisional U.S. Patent Application 61/745,212 to Dylan Van Hoven, filed on December 21, entitled “Method and Apparatus for Focusing Miniature Ultrasound Transducers”, and attorney docket 44755.1061, the contents of each which are herein incorporated by reference in its entirety. Since the transducer 210 is a micro-electrical mechanical system (MEMS) device, the substrate 200 may also be referred to as a MEMS substrate.

The micro-substrate 201 contains micro-electronic circuitry for controlling and interacting with the transducer 210. In the illustrated embodiment, such micro-electronic circuitry is implemented as an Application-Specific Integrated Circuit (ASIC) 220, where the micro-substrate 201 serves as a substrate for the ASIC 220. The ASIC 220 may be electrically coupled to the micro-substrate through conductive pads 230. It is understood that in other embodiments, the micro-substrate 201 itself may be an Integrated Circuit (IC) chip.

In the embodiment shown in FIG. 2, the substrate 200 including the transducer 210 is electrically and mechanically coupled to the substrate 201 including the ASIC 220 through wire-bonding. In more detail, the opposite distal ends of wire bonds (or bond wires) 225 are attached to bonding pads 230 on the substrate 200 and bonding pads 231 on the substrate 201, respectively. In some embodiments, the bonding pads 230-231 are smaller than about 60 um×60 um. The wire bonds 225 are electrically conductive and allow electrical communication to be established between the transducer 210 and the ASIC 220. In other words, the ASIC 220 can send electrical signals to, and/or receive electrical signals from, the transducer 210 to control and interact with the transducer 210. The wire bonds 225 are somewhat flexible and may allow the substrates 200 and 201 to be moved, rotated, or shifted with respect to one another to some degree. In some embodiments, the bonding loops are smaller than about 300 um in height. In some embodiments, the wire bonding is performed at temperatures less than about 70 degrees Celsius to avoid overheating the transducer 210 or the ASIC 220.

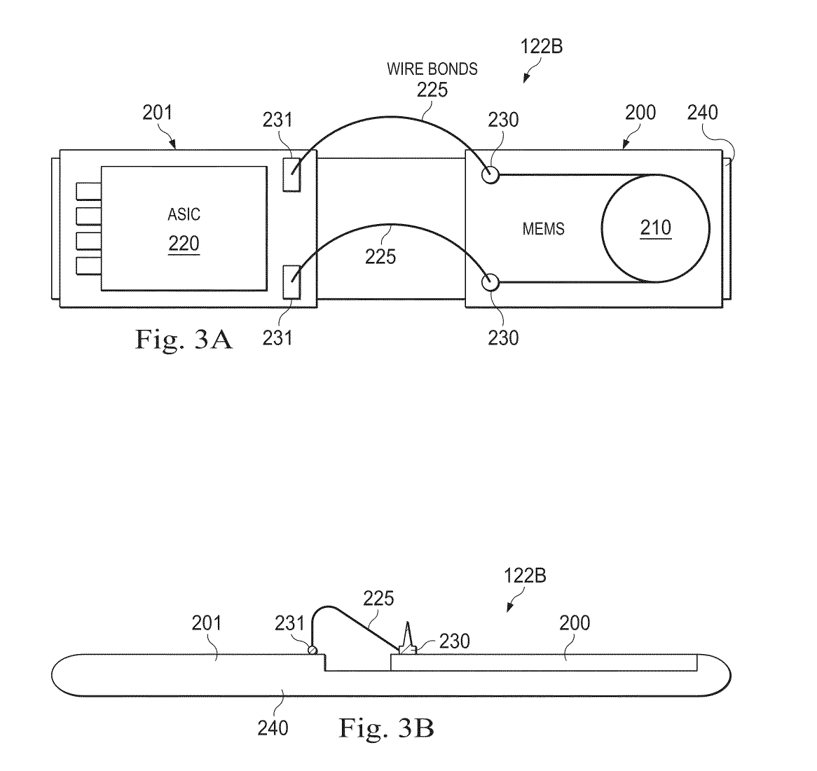

FIGS. 3A-3B are simplified diagrammatic top and cross-sectional views, respectively, of another embodiment of the transducer assembly 122B of the present disclosure. The embodiment of the transducer assembly 122B shown in FIGS. 3A-3B is similar to the embodiment of the transducer assembly 122A shown in FIG. 2. Therefore, for reasons of consistency and clarity, similar components in these two embodiments are labeled the same.

In more detail, the transducer assembly 122B also includes a substrate 200 (having the transducer 210) that is bonded to a substrate 201 (having the ASIC 220) through wire bonds 225. However, a support substrate 240 (also referred to as a supporting backing component) is attached to the substrates 200 and 201. As shown in FIG. 3B, the support substrate 240 supports the bottom sides of the substrates 200-201. Alternatively stated, the substrates 201-200 are disposed over or on the support substrate 240. The support substrate 240 provides mechanical strength and support for the substrates 200 and 201 disposed thereon.

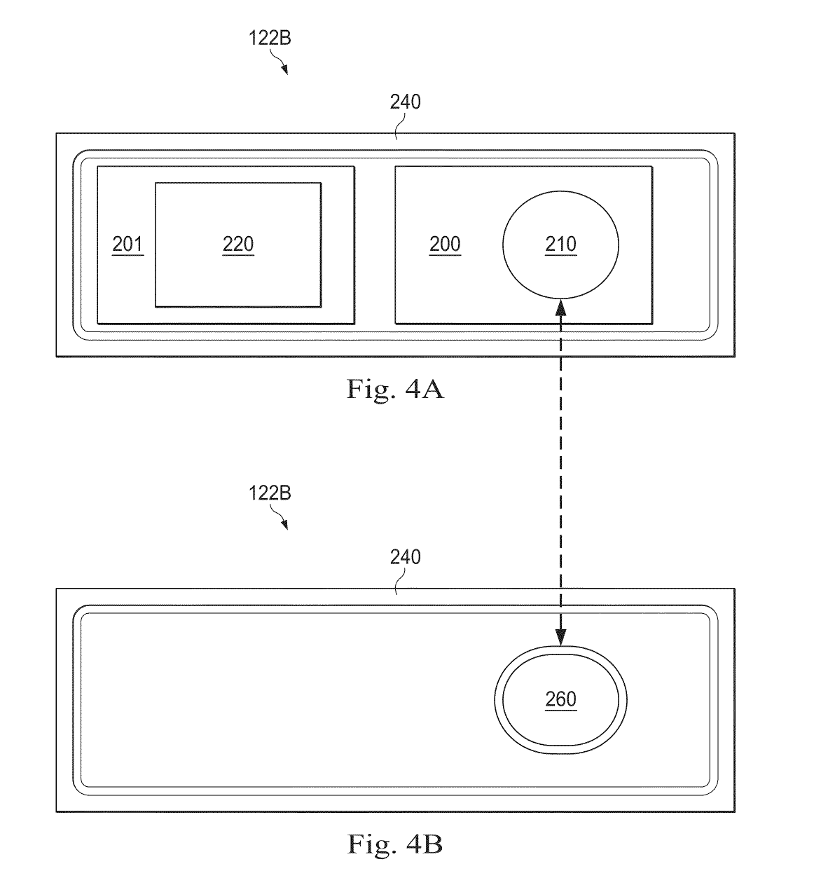

In some embodiments, an opening or hole may be formed in the support substrate 240 to expose the transducer 210. For example, FIG. 4B illustrates a bottom view of the transducer assembly 122B where an opening 260 (or hole) has been formed behind the transducer 210 in the back side of the support substrate 240. FIG. 4A is also provided alongside FIG. 4B, where FIG. 4A shows a simplified top view of the transducer assembly 122B to illustrate the positional placement of the opening 260 relative to the transducer 210. It is understood that in some embodiments, the support substrate 240 is a continuous piece with no openings or holes formed therein.

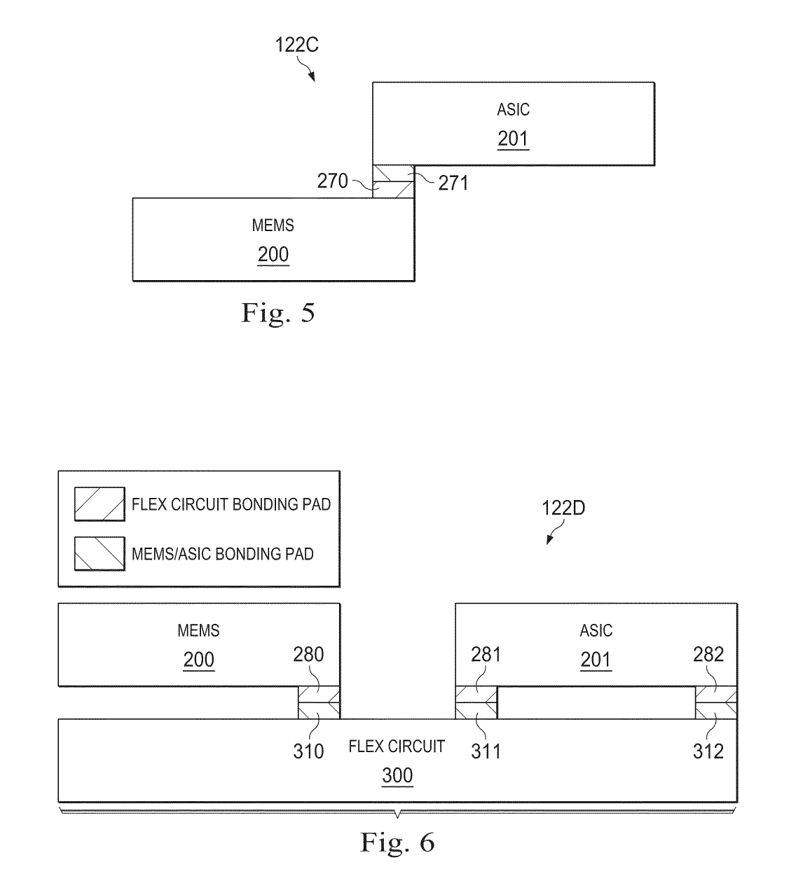

FIG. 5 is a simplified diagrammatic cross-sectional view of another embodiment of the transducer assembly 122C of the present disclosure. To the extent that the transducer assembly 122C of FIG. 5 is similar to the transducer assembly 122A shown in FIG. 2, similar components in these two embodiments are labeled the same.

In more detail, the transducer assembly 122C also includes a substrate 200 (having the transducer 210, which is not shown in FIG. 5 for reasons of simplicity) that is bonded to a substrate 201 (having the ASIC 220, which is not shown in FIG. 5 for reasons of simplicity) through flip-chip bonding. A conductive bonding pad 270 of the substrate 200 is bonded to a conductive bonding pad 271 of the substrate 201. Through the bonding pads 270-271, electrical communication between the transducer on the substrate 200 and the ASIC on the substrate 201 may be established. The bonded bonding pads 270-271 also mechanically hold the substrates 200-201 together.

FIG. 6 is a simplified diagrammatic cross-sectional view of another embodiment of the transducer assembly 122D of the present disclosure. To the extent that the transducer assembly 122D of FIG. 6 is similar to the transducer assembly 122A shown in FIG. 2, similar components in these two embodiments are labeled the same.

In more detail, the transducer assembly 122D includes a substrate 200 (having the transducer 210, which is not shown in FIG. 6 for reasons of simplicity), as well as a substrate 201 (having the ASIC 220, which is not shown in FIG. 6 for reasons of simplicity). The substrate 200 includes a conductive bonding pad 280, and the substrate 201 includes conductive bonding pads 281-282. Through these bonding pads 280-282, the substrates 200-201 are bonded to a flex circuit 300 through flip-chip bonding. Specifically, the flex circuit 300 includes conductive bonding pads 310-312, to which the bonding pads 280-282 are bonded, respectively. The flex circuit 300 is flexible and can be bent or “flexed” to conform to a desired shape. The flex circuit 300 itself may contain micro-electronic components and associated electrical routing, such as vias and metal lines (not shown herein for reasons of simplicity). Through the flex circuit 300, electrical communication between the transducer on the substrate 200 and the ASIC on the substrate 201 may be established.

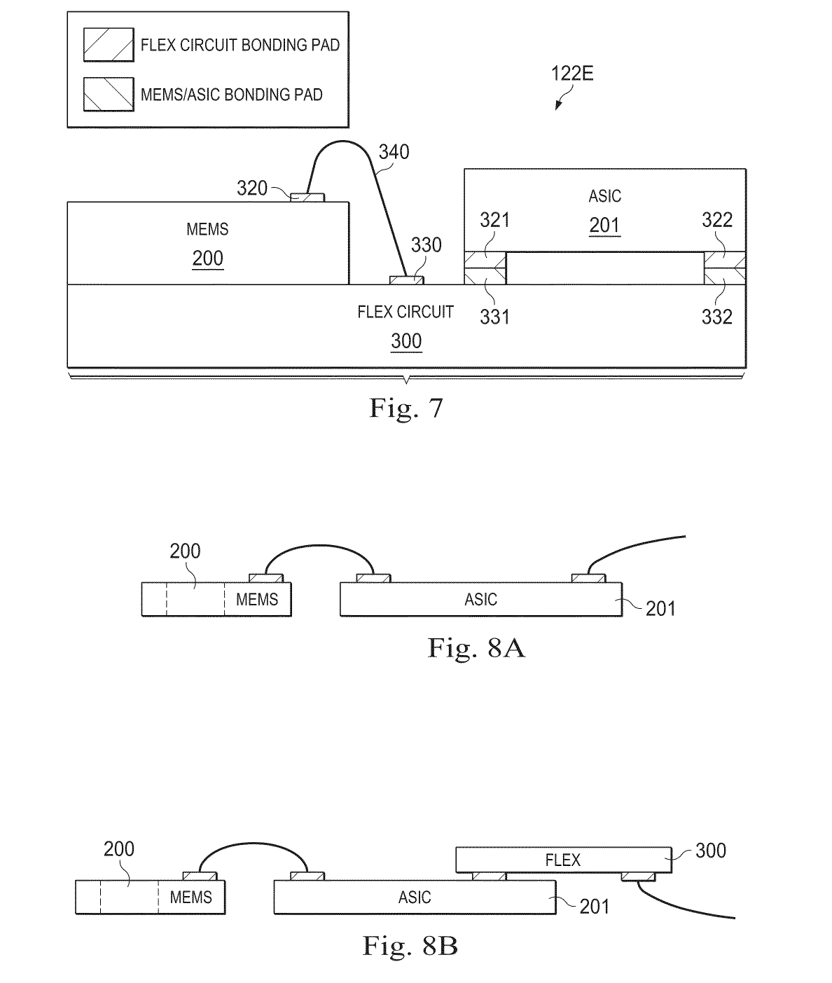

FIG. 7 is a simplified diagrammatic cross-sectional view of another embodiment of the transducer assembly 122E of the present disclosure. To the extent that the transducer assembly 122E of FIG. 7 is similar to the transducer assembly 122A shown in FIG. 2, similar components in these two embodiments are labeled the same.

In more detail, the transducer assembly 122E includes a substrate 200 (having the transducer 210, which is not shown in FIG. 7 for reasons of simplicity), as well as a substrate 201 (having the ASIC 220, which is not shown in FIG. 7 for reasons of simplicity). The substrate 200 includes a conductive bonding pad 320, and the substrate 201 includes conductive bonding pads 321-322. Through these bonding pads 320-322, the substrates 200-201 are bonded to a flex circuit 300. Specifically, the flex circuit 300 includes conductive bonding pads 330-332, to which the bonding pads 320-322 are bonded, respectively. In the embodiment shown, the substrate 200 is bonded to the bonding pad 330 of the flex circuit 300 through a wire bond 340 (or bond wire), and the substrate 201 is bonded to the bonding pads 331-332 of the flex circuit 300 through the flip-chip technology. In some alternative embodiments, the substrate 200 may be bonded to the flex circuit 300 through flip-chip, and the substrate 201 may be bonded to the flex circuit 300 through wire bonding. In yet other alternative embodiments, both the substrate 200 and the substrate 201 may be bonded to the flex circuit 300 through wire bonding.

Again, the flex circuit 300 is flexible and can be bent or “flexed” to conform to a desired shape. The flex circuit 300 itself may contain micro-electronic components and associated electrical routing, such as vias and metal lines (not shown herein for reasons of simplicity). Through the flex circuit 300, electrical communication between the transducer on the substrate 200 and the ASIC on the substrate 201 may be established.

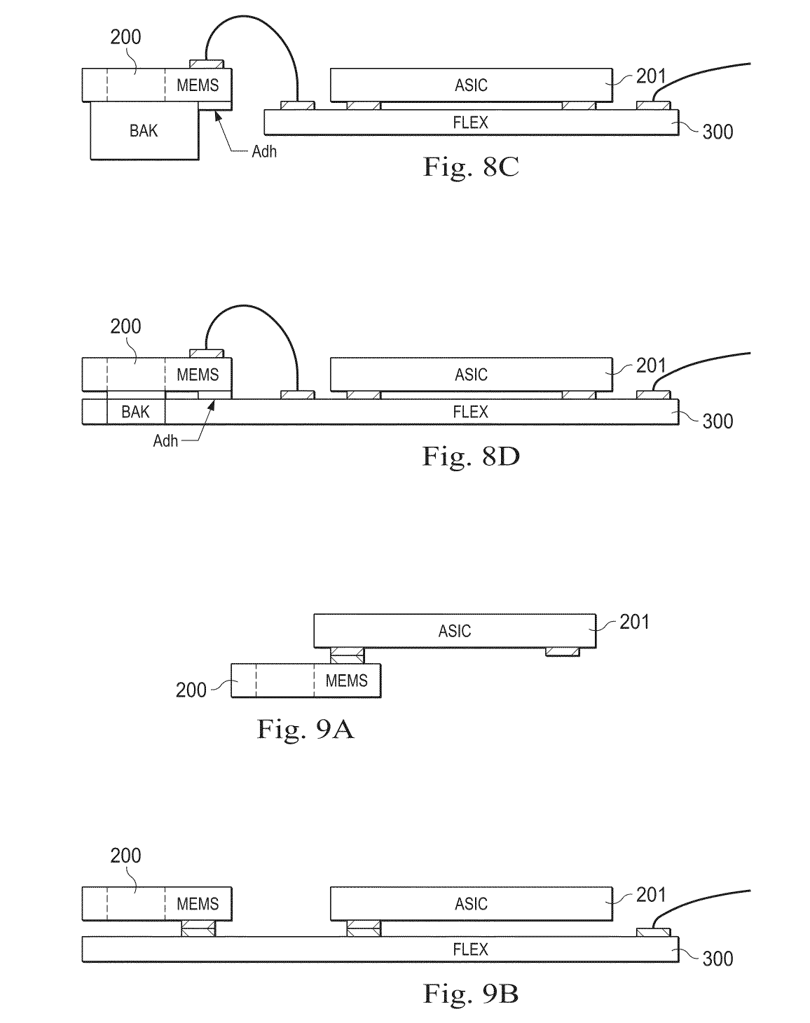

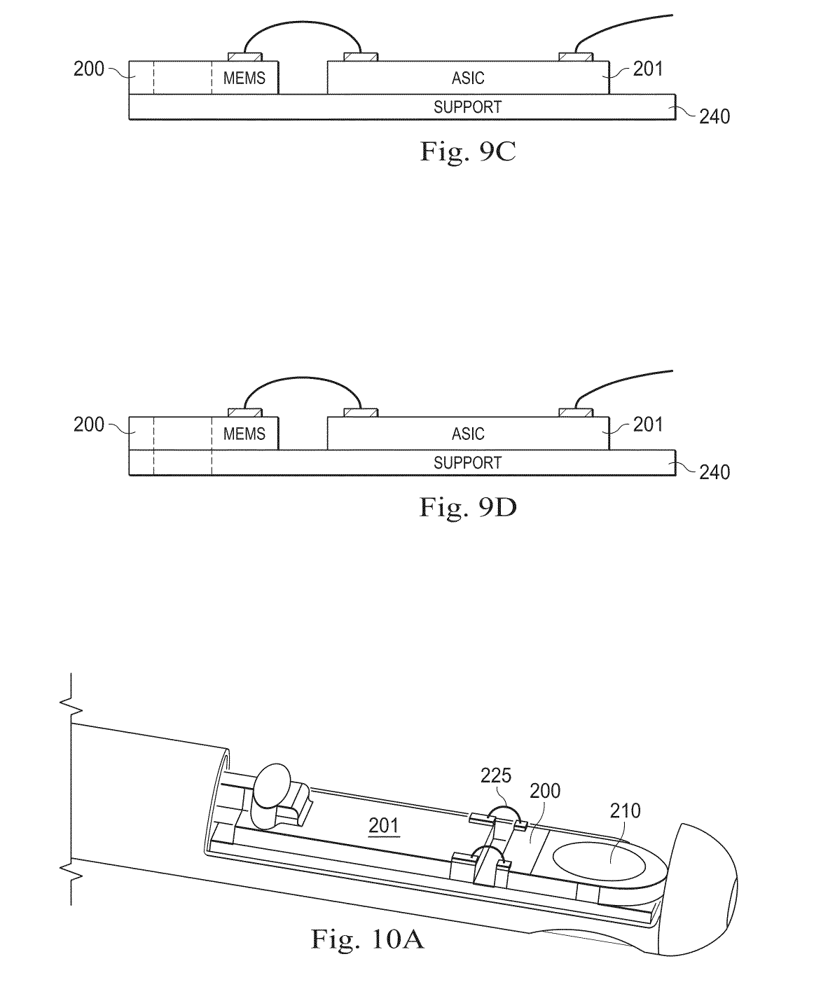

FIGS. 8A-8D and 9A-9D illustrate simplified cross-sectional views of various embodiments of transducer assemblies, some of which may be similar to those discussed above with reference to FIGS. 1-7. To the extent that the transducer assemblies illustrated in FIGS. 8A-8D and 9A-9D are similar to the transducer assemblies discussed above with reference to FIGS. 1-7, similar components are labeled the same for reasons of consistency and clarity.

In the embodiment shown in FIG. 8A, the substrates 200 and 201 are coupled together through wire-bonding. In the embodiment shown in FIG. 8B, the substrates 200 and 201 are coupled together through wire-bonding, and the substrate 201 is also coupled to the flex circuit 300 through flip-chip. In the embodiment shown in FIG. 8C, the substrate 200 is coupled to the flex circuit 300 through wire bonding, and the substrate 201 is coupled to the flex circuit through flip-chip. The flex circuit 300 does not provide support to the substrate 200 in this embodiment. In the embodiment shown in FIG. 8D, the substrate 200 is coupled to the flex circuit 300 through wire bonding, and the substrate 201 is coupled to the flex circuit through flip-chip. The flex circuit 300 does provide support to the substrate 200 in this embodiment.

In the embodiment shown in FIG. 9A, the substrates 200 and 201 are coupled together through flip-chip. In the embodiment shown in FIG. 9B, the substrates 200 and 201 are both coupled to the flex circuit 300 through flip-chip. In the embodiment shown in FIG. 9C, the substrates 200 and 201 are coupled together through wire-bonding, and they are both supported by a support substrate 240. The support substrate 240 in this embodiment does not have a through-hole. In the embodiment shown in FIG. 9D, the substrates 200 and 201 are coupled together through wire-bonding, and they are both supported by a support substrate 240. The support substrate 240 in this embodiment does have a through-hole.

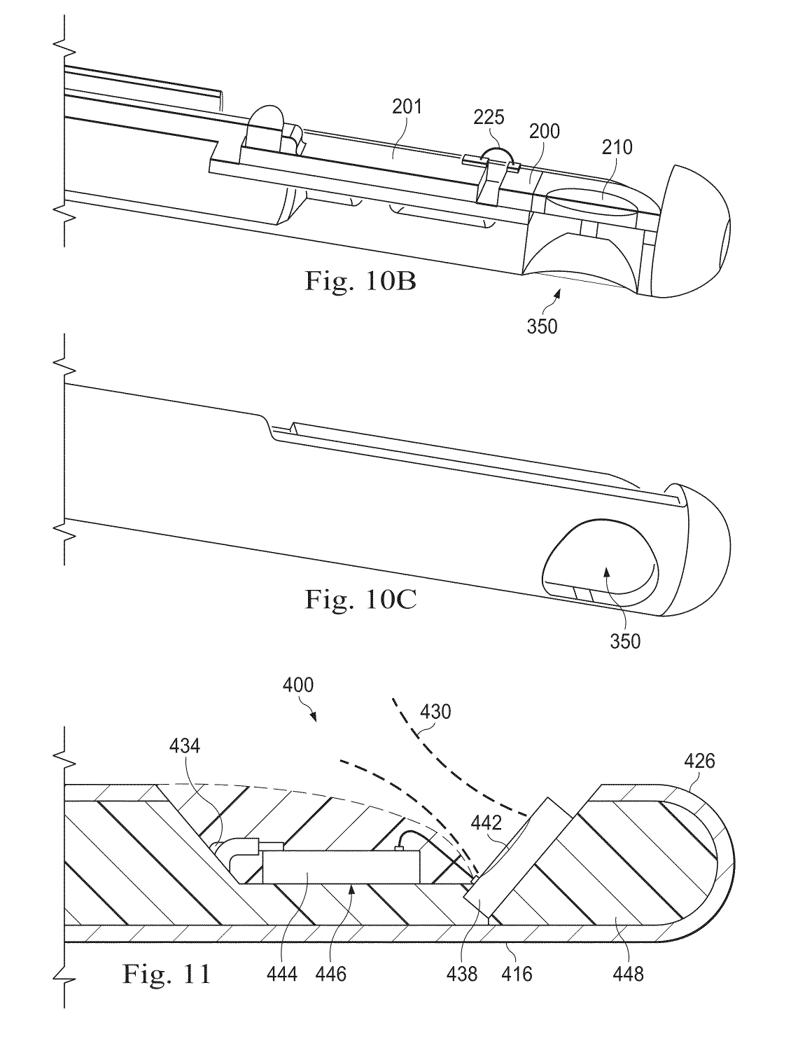

FIGS. 10A, 10B, 10C illustrate diagrammatic perspective views of an embodiment of a transducer assembly 122F from different viewing angles according to various aspects of the present disclosure. To the extent that the transducer assembly 122F of FIGS. 10A-10C is similar to the transducer assembly 122A shown in FIG. 2, similar components in these two embodiments are labeled the same.

In more detail, the transducer assembly 122F includes a substrate 200 having the transducer 210, as well as a substrate 201 (having the ASIC 220, which is not shown in FIG. 7 for reasons of simplicity). The substrates 200-201 are electrically coupled together through wire bonding, i.e., by wire bonds 225. Also, as can be seen in FIGS. 10B and 10C, a hole or opening 350 is formed to expose the transducer 210 on the back side. This hole or opening 350 may also be referred to as a well.

FIG. 11 illustrates a simplified diagrammatic cross-sectional view of an embodiment of an imaging core 400 that shows another embodiment of a transducer assembly, where the substrate having the transducer can be positioned at an angle with respect to the substrate having the ASIC. The substrate having the transducer is thereafter referred to as the MEMS 438, and the substrate having the ASIC is thereafter referred to as the ASIC.

As is shown in FIG. 11, the imaging core 400 includes a MEMS 438 having a transducer 442 formed thereon and an ASIC 444 electrically coupled to the MEMS 438. However, in the exemplary configuration of FIG. 11, the ASIC 444 and the MEMS 438 components are wire-bonded together, mounted to the transducer housing 416, and secured in place with epoxy 448 or other bonding agent to form an ASIC/MEMS hybrid assembly 446. The leads of the cable 434 are soldered or otherwise electrically coupled directly to the ASIC 444 in this embodiment.

One advantage of the wire-bonding approach is that the MEMS component carrying the transducer can be mounted at an oblique angle with respect to the longitudinal axis of the housing 416 and imaging core 400 such that the ultrasound beam 430 propagates at an oblique angle with respect to a perpendicular to the central longitudinal axis of the imaging core. This tilt angle helps to diminish the sheath echoes that can reverberate in the space between the transducer and the catheter sheath 412, and it also facilitates Doppler color flow imaging as disclosed in Provisional U.S. Patent Application No. 61/646,080 titled “DEVICE AND SYSTEM FOR IMAGING AND BLOOD FLOW VELOCITY MEASUREMENT” (Attorney Docket No. 44755.817/01-0145-US) and Provisional U.S. Patent Application No. 61,646,074 titled “ULTRASOUND CATHETER FOR IMAGING AND BLOOD FLOW MEASUREMENT” (Attorney Docket No. 44755.961), and Provisional U.S. Patent Application No. 61/646,062 titled “Circuit Architectures and Electrical Interfaces for Rotational Intravascular Ultrasound (IVUS) Devices” (Attorney Docket No. 44755.838), each filed on May 11, 2012 and each of which is hereby incorporated by reference in its entirety.

One aspect of the present disclosure involves a transducer assembly. The transducer assembly comprises: a first substrate that includes a piezoelectric micro-machined ultrasonic transducer (PMUT); and a second substrate that includes an Integrated Circuit (IC) device; wherein the first substrate and the second substrate are bonded together through wire bonding.

In some embodiments, the wire bonding is completed at temperatures below 70° C.

In some embodiments, the bonding pads are smaller than 60 um×60 um.

In some embodiments, the bonding loops are 300 um or smaller in height

One aspect of the present disclosure involves a transducer assembly. The transducer assembly comprises: a flex circuit; a first substrate that includes a piezoelectric micro-machined ultrasonic transducer (PMUT); and a second substrate that includes an Integrated Circuit (IC) device; wherein at least one of the first substrate and the second substrate is bonded to the flex circuit through wire bonding.

In some embodiments, the wire bonding is completed at temperatures below 70° C.

In some embodiments, the bonding pads are smaller than 60 um×60 um.

In some embodiments, the bonding loops are 300 um or smaller in height.

One aspect of the present disclosure involves a transducer assembly. The transducer assembly comprises: a support substrate; a first substrate that includes a piezoelectric micro-machined ultrasonic transducer (PMUT); and a second substrate that includes an Integrated Circuit (IC) device; wherein the first substrate and the second substrate are each bonded to the support substrate, and wherein the first substrate and the second substrate are electrically coupled together through wire bonding.

In some embodiments, the wire bonding is completed at temperatures below 70° C.

In some embodiments, the bonding pads are smaller than 60 um×60 um.

In some embodiments, the bonding loops are 300 um or smaller in height.

One aspect of the present disclosure involves a transducer assembly. The transducer assembly comprises: a first substrate that includes a piezoelectric micro-machined ultrasonic transducer (PMUT); and a second substrate that includes an Integrated Circuit (IC) device; wherein the first substrate and the second substrate are bonded together through soldering or welding.

In some embodiments, the bonding pads are smaller than 60 um×60 um.

One aspect of the present disclosure involves a transducer assembly. The transducer assembly comprises: a support substrate; a first substrate that includes a piezoelectric micro-machined ultrasonic transducer (PMUT); and a second substrate that includes an Integrated Circuit (IC) device; wherein the first substrate and the second substrate are each bonded to the support substrate, and wherein the first substrate and the second substrate are electrically coupled together through welding or soldering.

In some embodiments, the bonding pads are smaller than 60 um×60 um.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A transducer assembly, comprising:
a first substrate that includes a piezoelectric micro-machined ultrasonic transducer (PMUT); and
a second substrate that includes an Integrated Circuit (IC) device;
wherein the first substrate and the second substrate are bonded together through wire bonding.

2. The transducer assembly of claim 1, wherein the wire bonding is completed at temperatures below 70° C.

3. The transducer assembly of claim 1, wherein the bonding pads are smaller than 60 um×60 um.

4. The transducer assembly of claim 1, wherein the bonding loops are 300 um or smaller in height

5. A transducer assembly, comprising:
a flex circuit;
a first substrate that includes a piezoelectric micro-machined ultrasonic transducer (PMUT); and
a second substrate that includes an Integrated Circuit (IC) device;
wherein at least one of the first substrate and the second substrate is bonded to the flex circuit through wire bonding.

6. The transducer assembly of claim 5, wherein the wire bonding is completed at temperatures below 70° C.

7. The transducer assembly of claim 5, wherein the bonding pads are smaller than 60 um×60 um.

8. The transducer assembly of claim 5, wherein the bonding loops are 300 um or smaller in height.

9. A transducer assembly, comprising:
a support substrate;
a first substrate that includes a piezoelectric micro-machined ultrasonic transducer (PMUT); and
a second substrate that includes an Integrated Circuit (IC) device;
wherein the first substrate and the second substrate are each bonded to the support substrate, and wherein the first substrate and the second substrate are electrically coupled together through wire bonding.

10. The transducer assembly of claim 9, where in the wire bonding is completed at temperatures below 70° C.

11. The transducer assembly of claim 9, where the bonding pads are smaller than 60 um×60 um.

12. The transducer assembly of claim 9, where the bonding loops are 300 um or smaller in height.
